(19) United States
(12) Patent Application Publication
(10) Pub. No.: US 2021/0375155 A1
(43) Pub. Date: Dec. 2, 2021

(54) AUTOMATED COOKING ASSISTANT

(71) Applicants: Sarah Beth S. Brust, Edina, MN (US); Thomas Erik Brust, Edina, MN (US); Connor Richard Wray, St. Louis Park, MN (US)

(72) Inventors: Sarah Beth S. Brust, Edina, MN (US); Thomas Erik Brust, Edina, MN (US); Connor Richard Wray, St. Louis Park, MN (US)

(21) Appl. No.: 17/335,787

(22) Filed: Jun. 1, 2021

Related U.S. Application Data
(60) Provisional application No. 62/704,889, filed on Jun. 2, 2020.

(51) Int. Cl. G09B 19/00; A47J 47/00; A47J 36/32; G09B 5/02; G16H 20/60

(52) U.S. Cl. CPC G09B 19/003; A47J 47/005; G16H 20/60; G09B 5/02; G09B 19/0092; A47J 36/32

(57) ABSTRACT

An example cooking system can include: a cooking surface; a cooking device positioned adjacent to or within the cooking surface; and a projector associated with the cooking device, the projector being configured to project cooking instructions from the cooking device on the cooking surface. An example method for preparing food can include: selecting a cooking surface; positioning a cooking device adjacent to or within the cooking surface; and allowing the cooking device to project cooking instructions from the cooking device on the cooking surface.

### BACKGROUND

The internet has allowed people to find and share new or favorite recipes quickly and easily; however, the usability of these recipes in the kitchen setting is low. Mobile device screens have not been well integrated into the kitchen, as these displays often power down into power saving mode, require clean hands to navigate and use, and the recipes themselves are sometimes difficult to navigate between advertisements and single, large document formats.

### SUMMARY

The example automated cooking assistants described herein use a short-throw projector to display step-by-step recipe instructions, timers, techniques and tips, and overall progress directly onto a food prep surface. The assistants can integrate user feedback through touch feedback on the projection, allowing one or more users to easily navigate through the recipe while cooking.

### DESCRIPTION OF THE DRAWINGS

FIG. 1 shows the home screen of the cooking device of FIG. 17.

FIG. 2 shows the “Explore” screen of the cooking device of FIG. 17.

FIG. 3 shows the “Explore” functionality of the cooking device of FIG. 17.

FIG. 4 shows the “Cook” screen of the cooking device of FIG. 17.

FIG. 5 shows the “Cook” functionality of the cooking device of FIG. 17.

FIG. 6 shows the adaptive work area functionality of the cooking device of FIG. 17.

FIG. 7 shows the “My Profile” screen of the cooking device of FIG. 17.

FIG. 8 shows the “My Profile” functionality of the cooking device of FIG. 17.

FIG. 9 shows the multi-user screen of the cooking device of FIG. 17.

FIG. 10 shows the multi-user functionality of the cooking device of FIG. 17.

FIG. 11 shows the multi-recipe functionality of the cooking device of FIG. 17.

FIG. 12 shows the training screen of the cooking device of FIG. 17.

FIG. 13 shows line cook functionality of the cooking device of FIG. 17.

FIG. 14 shows the virtualization screen of the cooking device of FIG. 17.

FIG. 15 shows the Recipe Overview screen of the cooking device of FIG. 17.

FIG. 16 shows the My Recipe Book screen of the cooking device of FIG. 17.

FIG. 17 shows an example system including a cooking device.

### DETAILED DESCRIPTION

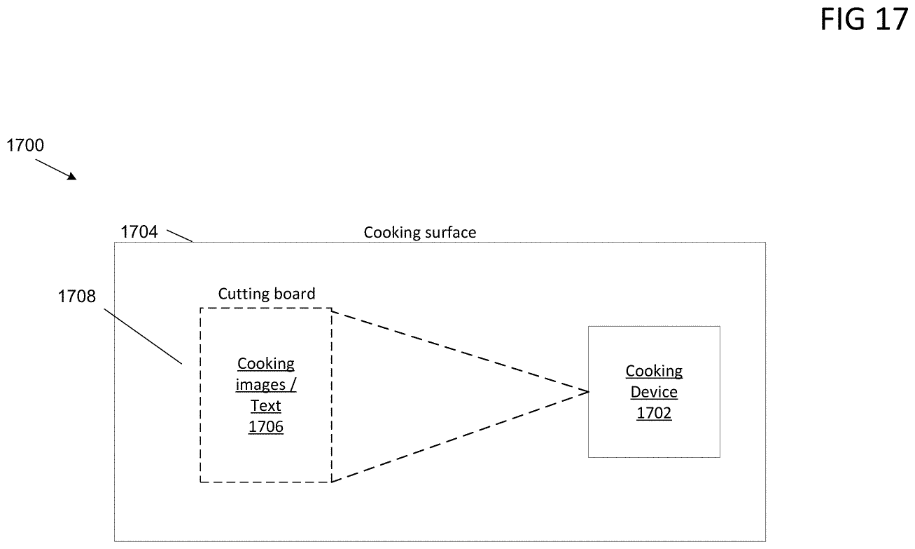

The example automated cooking assistants described herein streamline cooking by projecting intuitive, easy to navigate steps on an interactive food grade work surface. As shown in FIG. 17, the automated cooking assistants can be implemented in an example system 1700 including a cooking device 1702 positioned adjacent to or within a cooking surface 1704.

The entire display area for the device 1702 can have touch feedback, which is used to navigate between screens of the cooking device 1702, allowing users to engage in the different portions of the application. The device 1702 can use an ultra-short-throw horizontal projector to project high-resolution images 1706 directly onto the cooking surface 1704, which can be a washable food grade surface (e.g., a cutting board, tabletop, etc.) used for the preparation of food.

The device 1702 can emit an infrared light at the base of the device 1702, and an infrared camera of the device 1702 detects where the light beam is interrupted by human feedback to allow seamless interaction with the projected images 1706. There is also a visual light camera of the device 1702 that detects what is directly in front of the projector to enable the device 1702 to detect what a user is cooking, how much of an ingredient is in the work area to enable augmented reality cooking experiences.

A touch-enabled portion 1708 of the cooking surface 1704 changes based on the use for the work surface. During food preparation, the touch-enabled portion 1708 of the cooking surface 1704 can be reduced allowing users to use the other portions of the work surface for food prep creating an adaptive work area and avoiding object interference mis-clicks.

In examples provided herein, the device 1702 can be used to assist the user in preparation of one or more recipes. The device 1702 can project information associated with the recipes onto the cooking surface 1704. This information can be, for example, cooking instructions, suggestions on where to place food on the cooking surface 1704, etc.

The device 1702 can also sense items and/or the user's hands on the cooking surface 1704 to ascertain when certain aspects of the recipe have been performed. For instance, the device 1702 can sense when certain ingredients are positioned on the cooking surface 1704 (e.g., by sensing a generic item, size of an item, consistency of an item, and/or by matching image(s) of the item to known shapes, sizes, colors, etc. of the item). The device 1702 can also sense when the user's hands have performed aspects of the recipe, such as when items have been chopped, mixed, and/or placed or removed from the cooking surface 1704.

The device 1702 can use this information to control the presentation of the recipe to the user. For instance, the device 1702 can sense when a step of the recipe has been performed and automatically transitions to the next step without requiring explicit input from the user. The device 1702 can also provide suggestions to the user, such as indicating that an item has not been prepared and/or not been prepared correctly (e.g., indicating that an item has not been chopped finely enough for the recipe, etc.).

In alternative embodiments, the cooking device 1702 can include one or more displays built into the cooking device 1702 itself that display cooking information and allow the user to interact therewith. In yet other embodiments, the device 1702 can use a combination of projection and display on the device 1702 to present the recipe to the user. For instance, the steps of the recipe can be displayed on the device 1702, while specific instructions for a step are projected onto the cooking surface 1704. Other configurations are possible.

The device 1702 and associated cooking assistant application displayed thereby allow users to orchestrate food preparation for an entire meal by sequencing multiple recipe steps based on total time, active time, transition time, and passive time. The cooking assistant application stitches the instructions together by eliminating latent time overlaps across recipes ensuring the quickest overall cook time and that all meal items reach completion simultaneously. Additionally, the device 1702 and cooking assistant application offer recipes that are optimized to include a two-person side-by-side mode of instruction.

This functionality will separate recipe steps into two distinct task-lists for users to clearly and easily contribute to the same recipe together in real-time. The users of the device 1702 and cooking assistant application can fully customize their experience by setting up a profile which will influence the estimated cook and completion times, the suggested recipes, and the tips given in each recipe. The device's algorithm will automatically adapt to the user's cooking pace to adjust the user's profile.

The device 1702 and cooking assistant application can be used to find new recipes, execute multiple dishes in a meal, train and learn recipes and techniques, and cook with others through multi-user mode or video conferencing. The device 1702 and cooking assistant are cloud enabled and can interface with related services and platforms to support or enhance the cooking experience, including grocery shopping and delivery, nutritionist counseling, meal planning, nutritional breakdowns, music streaming, video streaming, “smart” kitchen appliances and their control systems, “smart” kitchen devices and their control system, and other application programming interfaces (“APIs”) that provide cooking, food, nutrition, or kitchen functionality.

Further, in some embodiments the device 1702 can be programmed to interface with other devices. For example, the device 1702 can be programmed to interface with the user's oven over a wireless connection. The device 1702 can thereby control the oven, such as by starting the oven at a certain baking type (e.g., bake, broil, etc.) and setting a timer for baking. The device 1702 can also communicate with the oven to determine when baking is complete and further configure the oven for other baking tasks and/or turn off the oven when complete. In another example, the device 1702 is programmed to communicate wirelessly with one or more volumetric or mass scales. This allows the device 1702 to automate the measurements (volume or weight) associated with the selection of ingredients for the recipe. Many other configurations are possible.

Generally, the cooking device 1702 includes at least one central processing unit (“CPU”) and computer-readable data storage media including a system memory. The system memory includes a random access memory (“RAM”) and a read-only memory (“ROM”). The system memory stores software instructions and data. The system memory is connected to the CPU. The system memory provides non-volatile, non-transitory storage for the cooking device. Computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the cooking device.

The computer-readable data storage media of the cooking device can store software instructions and data, including an operating system suitable for controlling the cooking device. The computer-readable data storage media also store software instructions and software applications that, when executed by the CPU, cause the cooking device to provide the functionality discussed herein.

More specifically, the hardware in the example cooking device 1702 can be a combination of a short throw projector, with an interactive touch surface where via cameras and other visual sensors interpret human touch. The initial device has internal storage and RAM, is Bluetooth and both 2.4 Ghz and 5 Ghz Wi-fi enabled. The application can pull both local user and favorited recipe data from the internal storage, but the device will primarily function via its wi-fi connection to connect to the full library of content available via virtual database storage. The device 1702 supports pushed updates so that the software can continue to be updated post-purchase.

There are built-in speakers and standard physical power and volume buttons on the device. There is also a microphone for voice activation and internet calls. For example, either or both of touch and voice activation can be used to control the device 1702, including navigation within a recipe, such as between steps of the recipe. The device 1702 has access to cooking assistant application content that includes live and recorded chef-led instructional classes, cooking and instruction configurations, customizable themes and skins, animation and musical themes, and curated recipes.

The device 1702 uses customized software that is loaded onto a short throw projection tower that projects images and videos onto a food-safe interactive surface. The projector uses visual feedback from the user to enable touch interaction and control the projection. The software supports the functions of step-by-step recipe guidance, exploring recipes, and instructions and training for both individuals and restaurants/organizations. The core function of the device 1702 and cooking assistant application, the cooking guidance, works by breaking down and encoding each step in terms of total time, active time, transition time, and passive time. The recipe management is done by creating a timeline that codes different portions of the recipe as active, passive and transition time based on the specifications for each recipe-active, passive, and transition time combined yields the total time.

As an example, if the user is making cookies and is prompted to combine the dry ingredients, the recipe would be coded as a single step with multiple actions. The total time “forecasted” would be four minutes and 10 seconds to combine the flour, sugar, baking soda, salt, and baking powder, with five active steps—each step is 20 seconds in length, 30 seconds of transition time between each step, and no latent time. Users would be prompted to add the required ingredients and they are able to indicate that they had completed each task within the cooking assistant application. Users will be able to swipe to proceed to the next cooking step once they have completed the displayed instructions regardless of how many steps the user has marked as complete. The program records how fast the user moves between both active and passive steps updating the user profile to improve future time predictions.

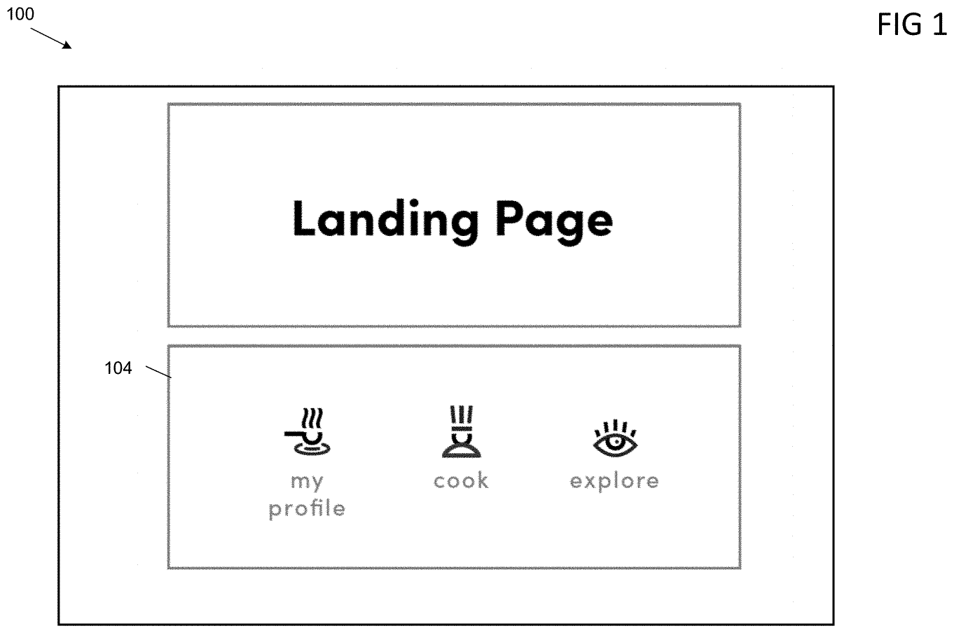

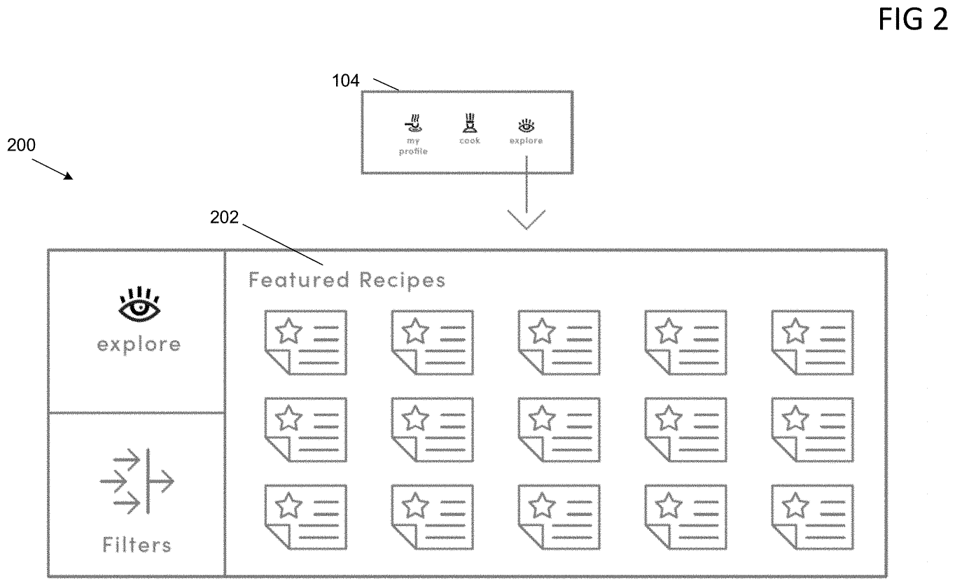

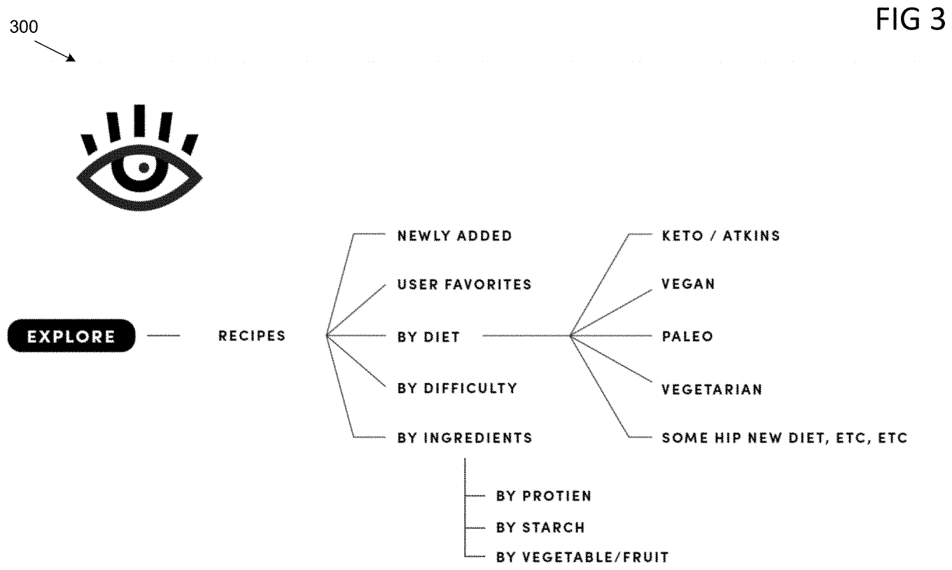

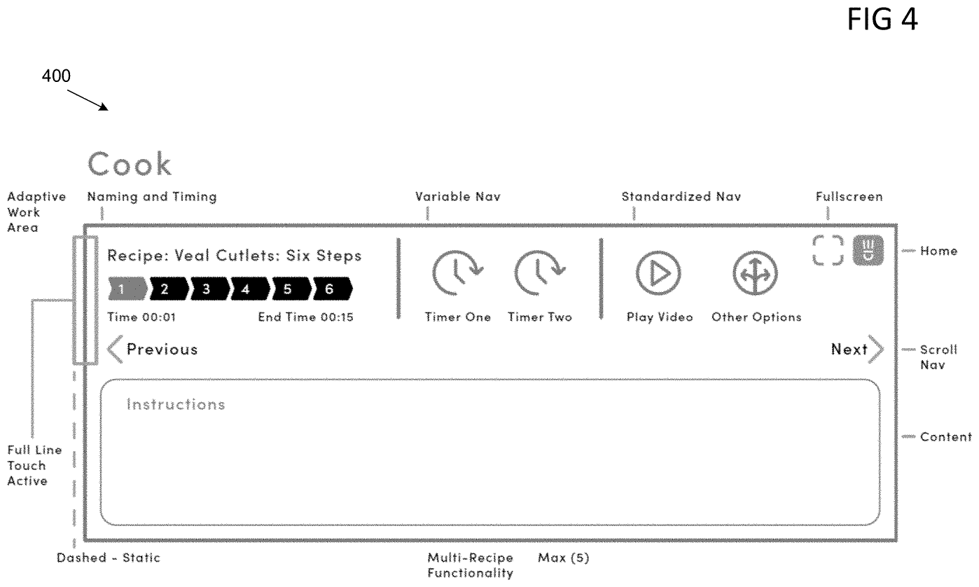

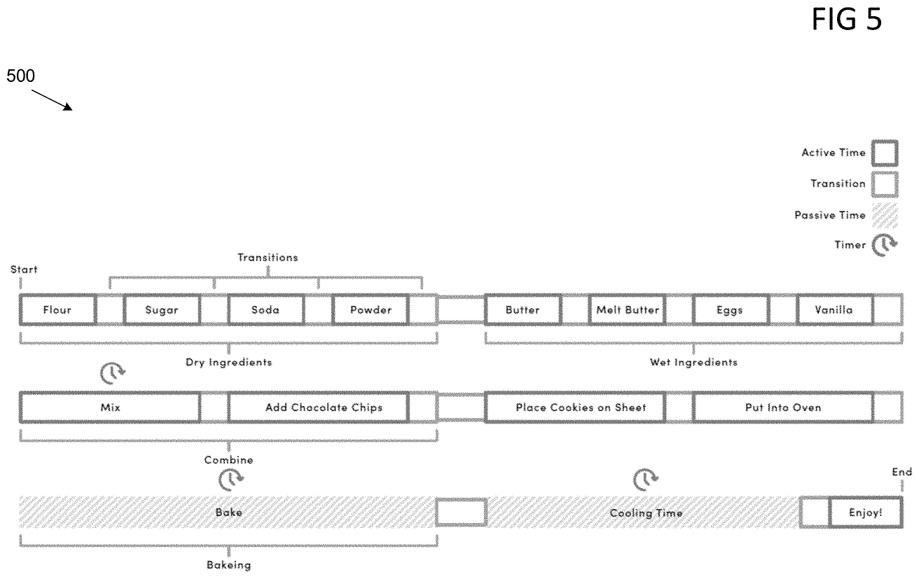

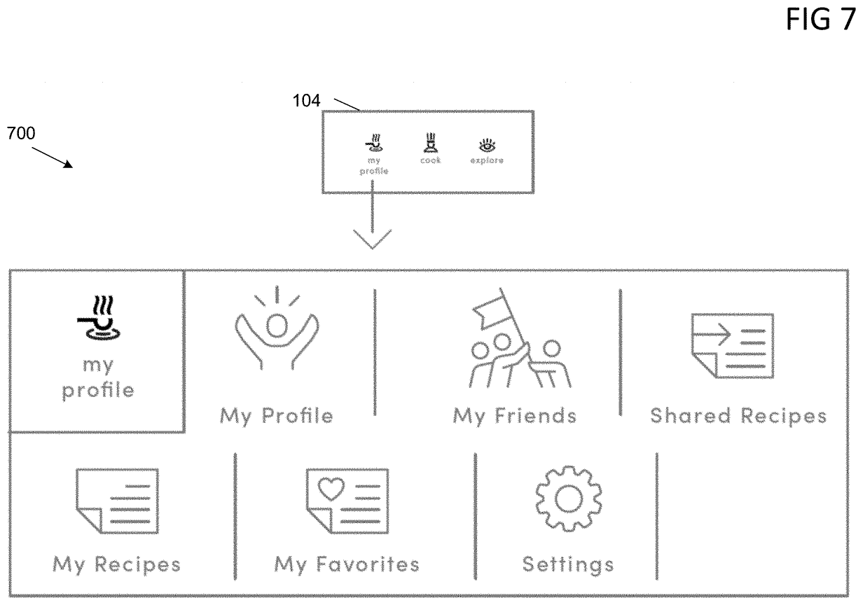

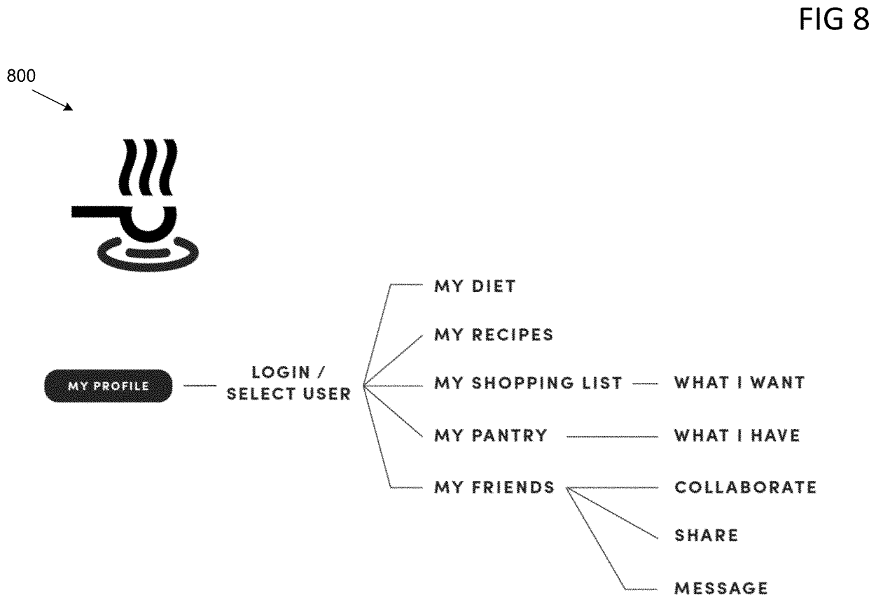

FIG. 1 shows an example of what options 104 will be available to the user upon startup of the device 1702. These options 104 on a screen 100 will be to engage in the explore screen 200 (see FIGS. 2-3), cook screen 400 (see FIGS. 4-5), and my profile screen 700 (see FIGS. 7-8) of the device 1702. The device 1702 loads one of these options upon selection by the user, either using a pointing device, touch, and/or voice activation on the device 1702.

FIG. 2 shows the explore screen 200 and FIG. 3 describes the explore functionality 300. In “Explore,” users may search through a library 202 of food and drink recipes filtering and sorting by category, ingredient, cook time, diet, reviews, ingredients, difficulty, chef or creator of the recipe, recommended for kids, or nutritional information (among other filters). Each recipe is represented by a photo of the completed dish and the recipe title followed by a few short statistics about the dish represented by either icons or a few words. From here, users can add recipes to their my recipe book screen 1600 through clicking a favorite button. The program saves each of the favorited recipes to a user profile database.

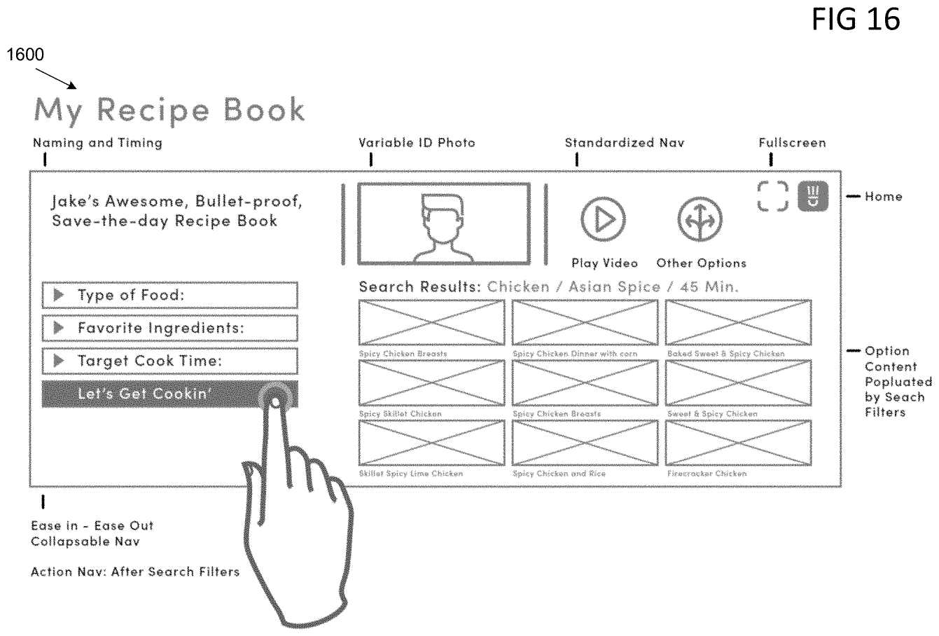

FIG. 16 shows the my recipe book screen 1600. The my recipe book screen 1600 can be accessed from the explore screen 200, my profile screen 700, and cook screen 400. Each recipe is indexed by filtering categories and has a unique identification number. Selecting the recipe will bring up a quick preview screen allowing the user to read more details about the recipe including the ingredients, cook time, diet, reviews, and nutritional information.

FIG. 4 shows the cook screen 400 and FIG. 5 describes the cooking functionality 500. The cook screen 400 guides users through creating a dish by breaking the recipe into intuitive steps with interactive instructional animations and videos supplementing short written instructions. The animation and videos are saved to the unique recipe ID and are loaded into temporary active device memory for quick access once the user begins cooking from the overview screen.

The cooking functionality 500 depicts various timelines that represent steps and/or ingredients used in the recipe(s). A first timeline represents ingredients that are used at different steps of the recipe, along with transitions between each ingredient. A second timeline represents actions to be performed, such as mixing, placing items on a sheet, and placing items in the oven. A third timeline represents the overall tasks to be performed for the recipe, such as baking, cooling, etc.

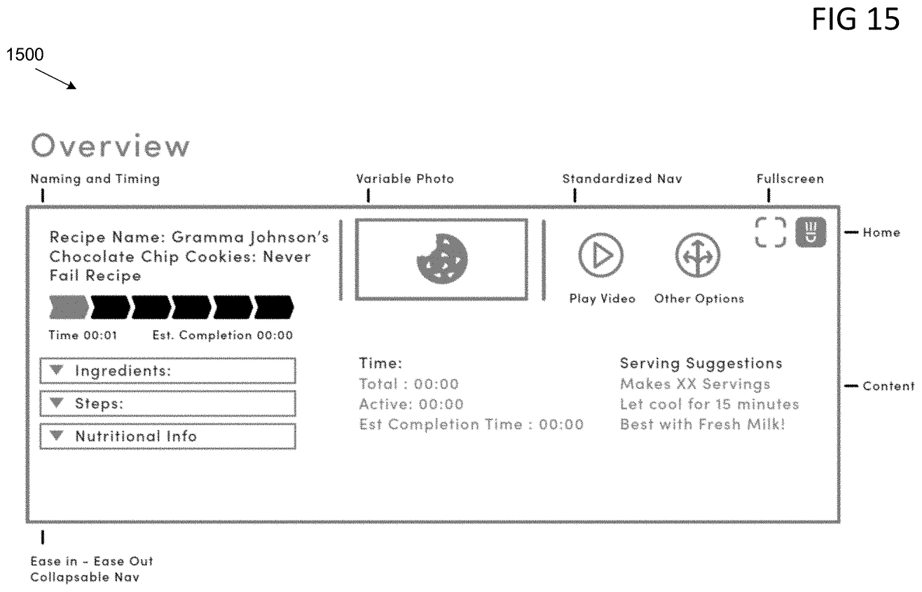

The user begins cooking a recipe by selecting a recipe from the my recipe book screen 1600. The my recipe book screen 1600 displays a preview of the recipe, and the user selects “Recipe Overview” to begin cooking. FIG. 15 shows the recipe overview screen 1500. From the recipe overview screen 1500, the user can scale the recipe to various sizes and see the associated nutritional information and number of servings. The device 1702 saves each recipe as ratios among variables such as cook time and ingredients. The device 1702 scales recipe ingredients by direct ratios and alters other settings such as cook time and temperature by high-level recipe scaling settings. Users will also have the option to cook entirely by weight.

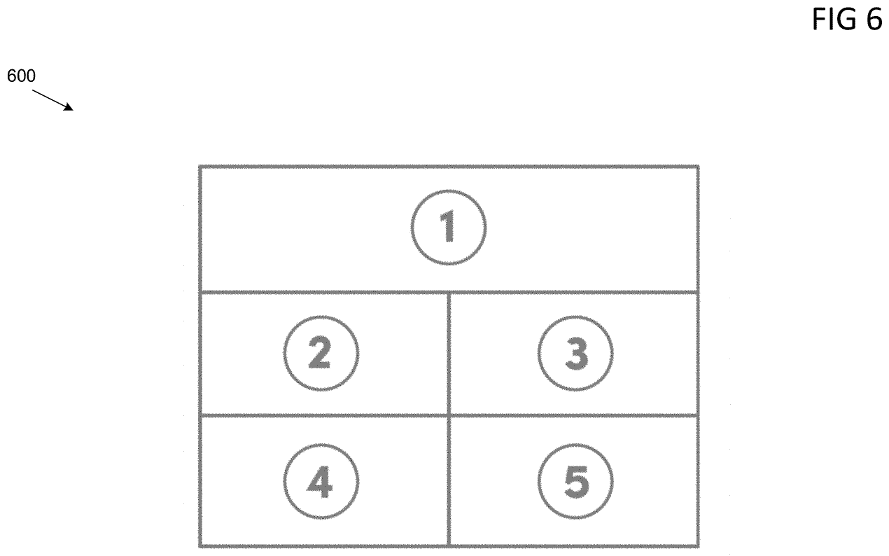

FIG. 6 illustrates the adaptive work area functionality 600. The projected image for the adaptive work area functionality 600 is broken into segments based on the screen that is displayed, certain segments are enabled for touch feedback while others may not be enabled. The work surface 1704 is intended to be used as a key portion of the meal prep, so as the user departs from the overview page, the portion of the screen that is touch enabled is reformatted to only the top of the screen so the bottom can be used for food prep if desired. The bottom portion of the newly divided screen is available for both food prep and projecting instructions and videos on the static work surface 1704 with or without touch feedback. If desired, the user will have the ability to enter full screen mode in the cook screen 400 to enable touch on the entire work surface 1704.

As the user begins the recipe, the cook screen 400 displays a tracker with an estimated completion time, the current step instructions, and a photo, video, and/or animation for how to do the step. The user proceeds through the steps by swiping left (or through voice activation), and he or she can return to previous steps by swiping right. Recommendations for timers and oven settings will appear on the appropriate steps. Users may set multiple timers, and timers will be displayed at the top of the screen allowing the user to continue to execute the recipe. Additionally, tips and suggestions will be displayed throughout the execution of the recipe such as plating suggestions and drink pairings for the meal being prepared. Once the recipe is completed, users are asked to rate/review the recipe, and they also have the option to add comments and personal notes for their reference. Additionally, users will see any awards that they have earned from completing the recipe, such as “First Recipe Cooked.”

The final option on the home screen is the my profile screen 700. FIG. 7 shows the my profile screen 700 screen and FIG. 8 shows the my profile functionality 800. The my profile screen 700 is where the personalization of the application takes place. Here, users can set up profiles including their dietary preferences and ingredient staples that they always have on hand in their home. Additionally, users will be able to fill out a profile with their photo, friend list, security and privacy settings, along with allergies, diet information, and serving size preferences. These profile settings are used to optimize the user experience through filtering recipes to reflect the user's preferences and tailoring the user experience while cooking. Furthermore, users can view a shopping list for any recipes they have added to their meal plan for the week. They can also manually enter or take photos of their existing physical recipe cards to record into their saved recipes to use on the device and share their favorite dishes with their friends and the other users in the community.

Users can build a network of other device users through searching for and sending/accepting friend requests. This allows users to see what their friends are making, and quickly share recipes, images, and notes. The my profile screen 700 will give users the ability to integrate nutrition information from the recipes that they cook with the device with fitness trackers to help users monitor their overall health. The my profile screen 700 also allows users to view any awards and achievements that they have earned through their activity using the device.

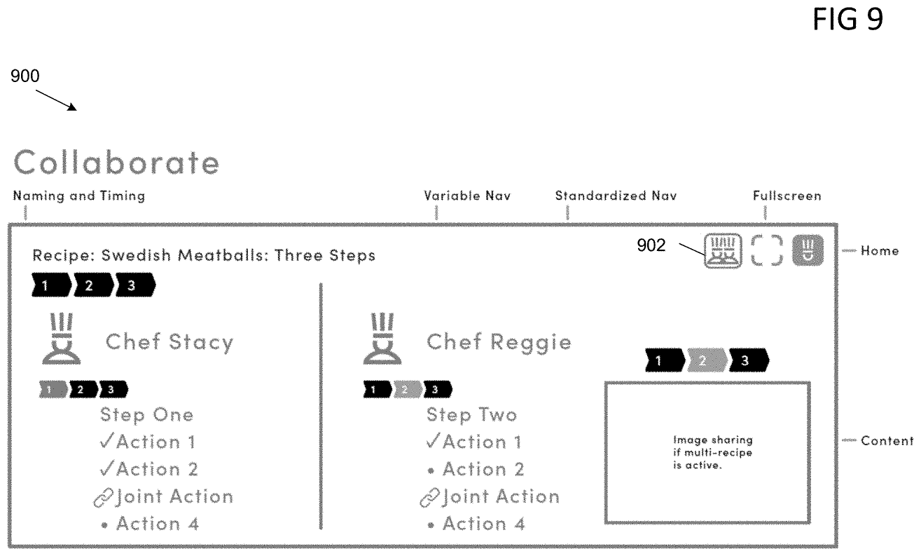

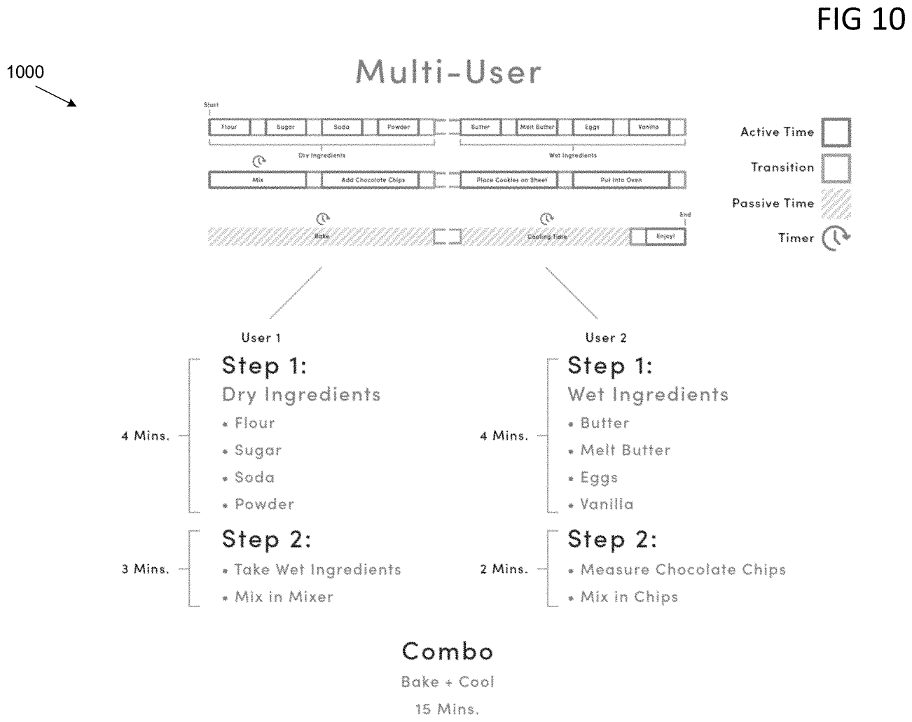

FIG. 9 shows the multi-user screen 900 and FIG. 10 shows the multi-user functionality 1000. Certain recipes will be marked as available for multiple users with a multi-user icon on the recipe card. These recipes will be optimized to include a two or more-person side-by-side mode of instruction. The program separates recipe steps into distinct task-lists for users to clearly and easily contribute to the same recipe together. The program looks at the total timeline for an entire recipe. It will separate groups of tasks, such as adding dry ingredients for the cookies, and assign them to a specific user.

All recipe steps entered into multi-user screen 900 will also be broken into active, passive, transition, and total time. The multi-user screen 900 works by breaking the active time into intuitive task lists where each user works on a certain discrete portion of the recipe. In each list of directions, there will be additional steps and notes indicating when the users need to interact combining their portions of the recipe.

When a user selects the multi-user screen 900 for a recipe, the display of the device 1702 will indicate that it is in multi-cook mode by showing the multi-cook icon 902. Once the mode is selected, the screen will split into several columns with one column for each user. Each column will display each user's individual list of instructions and timers. Each user will be able to advance through the steps of the recipe independently until the required shared steps are reached. When one user inevitably reaches the shared step first, they will be given suggestions such as tidy the work area and do dishes that are no longer needed if available. The shared steps will be combined into one column.

Multi-user mode may incorporate the individual user profiles to optimize parent and child collaboration or different cooking levels among collaborating users. Additionally, users who finish their steps can help with the other user's list to help move the recipe forward.

The device 1702 delivers the ability to combine multiple recipes into an integrated meal. Inside the my recipe book screen 1600, users can combine different recipes and portion sizes into a complete meal. The user will be able to select portions per recipe and nutritional information will be tracked individually. The my recipe book screen 1600 also includes suggested meals and a step by step meal builder and meal planner. Drink pairings as well as appropriate pans, utensils, and accessories will be suggested to the user based in his or her saved recipes. The device 1702 will allow users to act on these suggestions through choosing to make a suggested recipe or purchasing the suggested item.

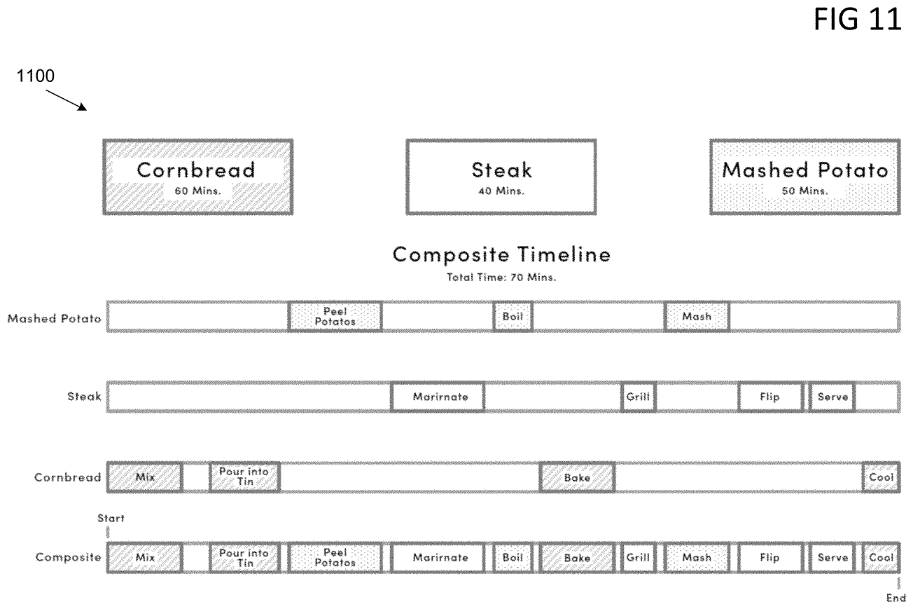

The device 1702 allows users to easily execute an entire meal and integrates many recipe steps from different dishes into a single set of steps to orchestrate an entire meal through the multi-recipe functionality. FIG. 11 shows the multi-recipe functionality 1100. Each recipe is mapped with a timeline with active, passive, transition, and total time periods. The device stitches the instructions together by splicing in active steps from other recipes during passive time from previously completed steps. The stitching of multiple recipes is shown on a single timeline at the bottom of the screen. An integrated tracker at the bottom of the screen will track the user's progress toward the completed meal. The tracker is an adaptive multi-axis progress bar that helps visualize coordinating multiple recipes such that they are done at the same time and can be served warm.

Meal steps adapt and learn the user's cooking pace by recording the speed in which they are moving between various steps and output an estimate of the time required to cook a dish. For instance, the device 1702 can sense how long it takes for the user to perform certain tasks and adapt the presentation of the recipe based upon this information. For example, the device 1702 can determine how long it takes the user to chop an ingredient and present future steps associated with chopping accordingly (e.g., more quickly or slowly), assuming a similar cadence from the user.

Additionally, users will be able to view and alter their preferred cooking pace in their my profile screen 700 settings to feed into the device's meal execution planning such that those who prefer a more relaxed meal preparation will receive different feedback and planning than those who prefer fast-paced meal execution.

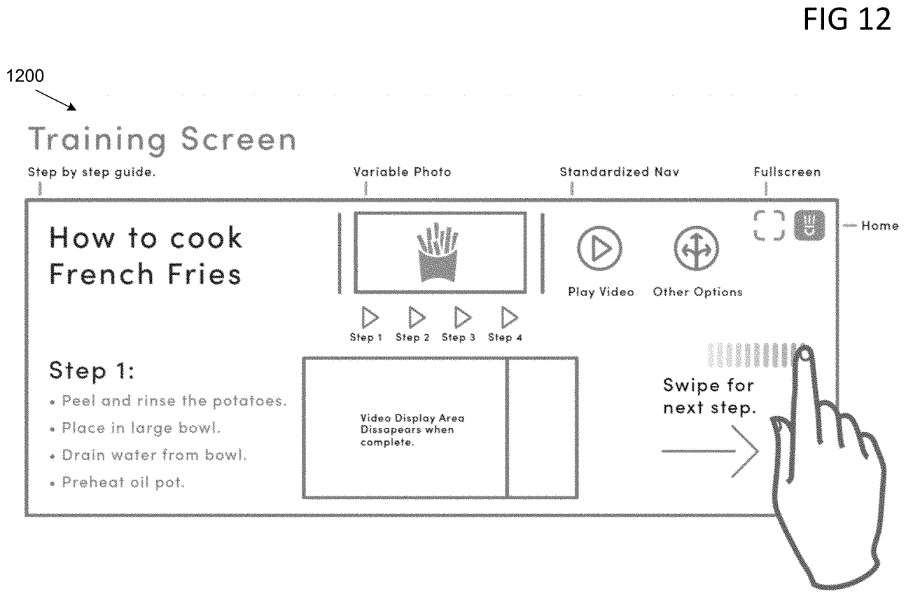

The explore screen 200 will include featured recipes and courses for users to take to learn particular cooking techniques or recipes. FIG. 12 shows the training screen 1200. The training screen 1200 has courses including more-detailed instructions, embedded videos and GIFs, and diagrams to assist the user in learning a new skill or recipe. Featured and favorited recipes will be highlighted at the top of the explore screen 200 for users. This featured content can be targeted at users based on their user profile and interests. Additional content such as the training and classes will be displayed on the top of the my recipe book screen 1600.

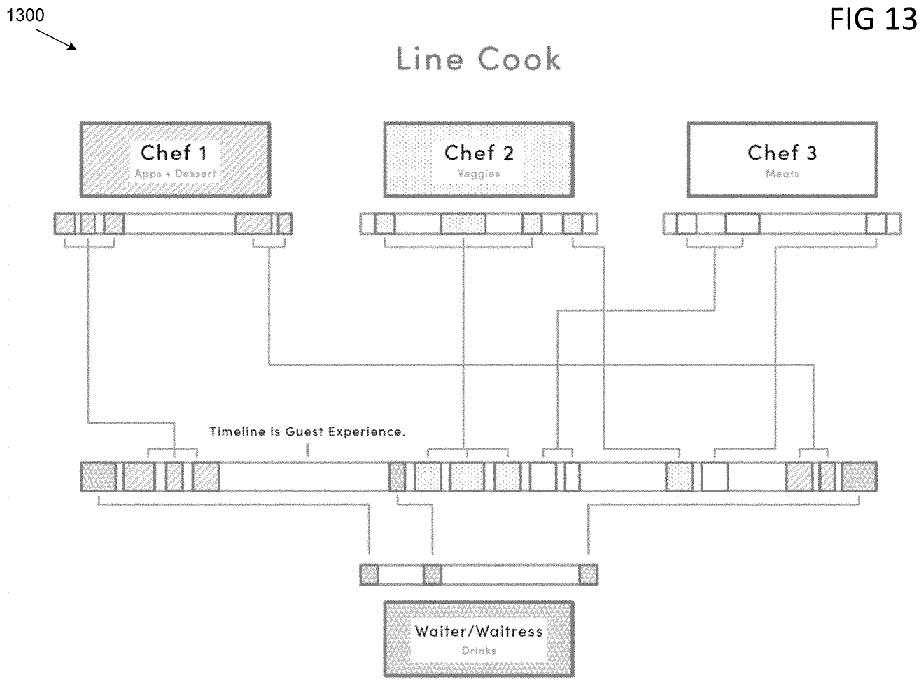

The device's cook functionality can be applied in an industrial kitchen environment to train line cooks as restaurant recipes can be loaded into the my recipe book screen 1600 and incoming orders through the point of sale systems will auto-modify recipes to reflect customer specifications. FIG. 13 shows line cook functionality 1300 which ensures that restaurant recipes are made using the correct steps and that modifications are made to the recipes according to customer orders. The line cook functionality 1300 provides a timeline for each chief to prepare certain portions of one or more meals, and these timelines are integrated into a master timeline that combines the steps of the different chefs. The master timeline can, in turn, be synchronized with the wait staff so that completed meals are delivered in a timely fashion.

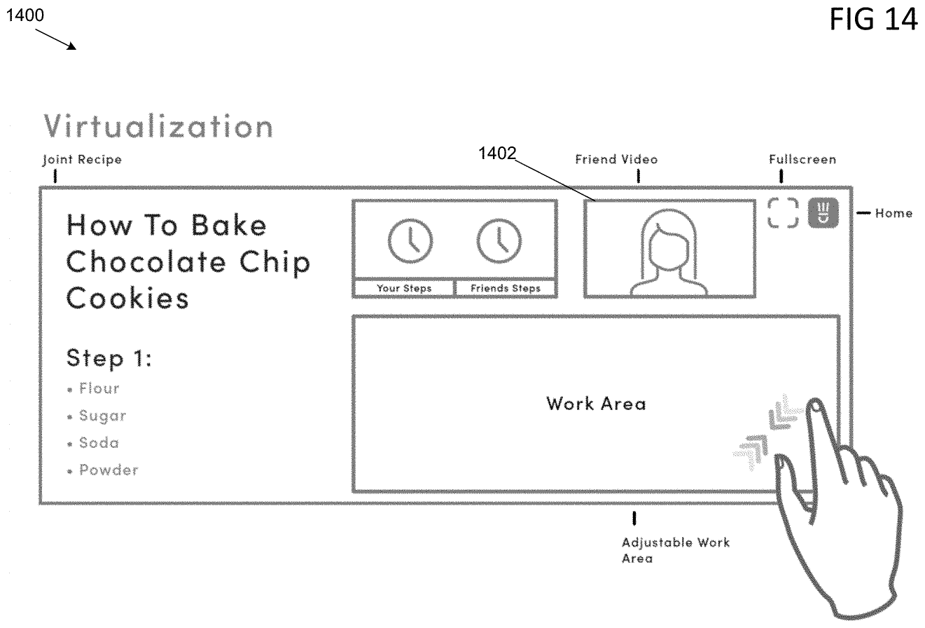

The device includes the option to video call and create the same recipe with another user virtually. FIG. 14 shows the virtualization screen 1400. In the my profile screen 700, a user can choose to video call another remote user (e.g., a user at a different location, such as another city, state, or country) who can accept the call, and then a video chat will be displayed on a portion 1402 of the virtualization screen 1400 while the two users use their devices to explore new recipes or cook a recipe together. For instance, the virtualization screen 1400 can allow to remote users to prepare the same recipe at the different locations. The video call functionality can also be used to give users to access expert coaching for help with a specific recipe or technique in real-time.

The device 1702 will have companion mobile and web interfaces such that users can access recipes and device functionality from their mobile devices and computer.

What is claimed is:

1. An example cooking system, comprising:
a cooking surface;
a cooking device positioned adjacent to or within the cooking surface; and
a projector associated with the cooking device, the projector being configured to project cooking instructions from the cooking device on the cooking surface.

2. The system of claim 1, wherein the cooking device is programmed to divide a recipe into multiple parts for multiple users to complete the recipe.

3. The system of claim 1, wherein the cooking device is programmed to integrate multiple recipes into a meal by sequencing each of the multiple recipes.

4. The system of claim 1, wherein the cooking device is programmed to learn a cadence and preferences of a user over time.

5. The system of claim 1, wherein the cooking device is programmed to provide training instructions for training a user.

6. The system of claim 1, wherein the cooking device is programmed to allow a user to video chat with a remote user.

7. The system of claim 6, wherein the cooking device is programmed to allow the user and the remote user to complete a recipe.

8. The system of claim 1, wherein the cooking device is programmed to integrate with a health system to track nutritional information.

9. The system of claim 1, wherein the cooking device is programmed to integrate with a shopping list generated based upon one or more selected recipes.

10. The system of claim 1, wherein the cooking device is programmed to integrate with other devices to complete cooking tasks or relay cooking information.

11. The system of claim 1, wherein the cooking surface is a food grade surface.

12. A method for preparing food, the method comprising:
selecting a cooking surface;
positioning a cooking device adjacent to or within the cooking surface; and
allowing the cooking device to project cooking instructions from the cooking device on the cooking surface.

13. The method of claim 12, further comprising dividing a recipe into multiple parts for multiple users to complete the recipe.

14. The method of claim 12, further comprising integrating multiple recipes into a meal by sequencing each of the multiple recipes.

15. The method of claim 12, further comprising learning a cadence and preferences of a user over time.

16. The method of claim 12, further comprising allowing a user to video chat with a remote user.

17. The method of claim 16, further comprising allowing the user and the remote user to complete a recipe.

18. The method of claim 12, further comprising integrating a health system to track nutritional information.

19. The method of claim 12, further comprising generating a shopping list based upon one or more selected recipes.

20. The method of claim 12, further comprising communicating with other devices to complete cooking tasks or relay cooking information.
